(19) United States
(12) Patent Application Publication
(10) Pub. No.: US 2003/0215511 A1
(43) Pub. Date: Nov. 20, 2003

(54) Fabrication of protein gel for new application

(76) Inventor: Jiunn Yeong Teo, Fayetteville, AR (US)

Correspondence Address: TEO, JIUNN YEONG, 2902 N. Gregg St. # 6, Fayetteville, AR, 72703, US

(21) Appl. No.: 10/147,090

(22) Filed: May 14, 2002

(51) Int. Cl. G01N 33/53; C07K 1/32; A61K 9/14; G01N 33/543

(52) U.S. Cl. 424/484; 435/7.1; 436/518; 530/402

(57) ABSTRACT

This invention relates to a method of forming protein gel matrices for separation, immobilization, diagnosis, and artificial organs. These matrices are made of proteins and are different from their polymeric counterparts. The formation of supported or non-supported flat sheet membranes, supported or non-supported tubular membranes, hollow fiber membranes, monoliths for continuous bed chromatography, enzyme-linked immuno sorbents, gas sorbents, liquid sorbents, edible sorbents, hemoperfusion sorbents, and surgical sponges are also disclosed.

### BACKGROUND OF THE INVENTION

1. Field of the Invention

The invention relates to the formation of protein gel matrices for separation, immobilization, diagnosis, and artificial organ applications in chemical, biochemical, and other industries. In particular, protein gels with different morphologies, characteristics, size, and shape suitable for separation, immobilization, diagnosis, and artificial organs are sought for in this invention.

2. Description of Prior Art

Protein has been utilized as a food and medicine for a long time, and relatively recently, protein gels have also been utilized as a material for food coatings, varnishes, and biodegradable plastics. This invention further expands the utilization of protein gel into separation, immobilization, diagnosis, and artificial organ applications common to chemical, biochemical, and other industries.

Related knowledge about the making of protein gel with different morphologies suitable for separation can be learned from the prior art related to the chemistry of heat induced protein gelation [Mulvihill and Kinsella, 1988; Ju and Kilara, 1998; Barbut, 1995]. The formation of a heat induced porous protein gel is a two-step process involving a change in the conformation of the protein molecules (denaturation) and the subsequent association (aggregation) of the denatured protein molecules to form porous aggregate gel. Network association involves reactions such as hydrogen bonding, ionic and hydrophobic interactions, and disulfide cross-linking. Disulfide bonding and the thiol-disulfide interchange reactions are the primary factors of producing a stronger thermoset gel, which is useful for separation and immobilization applications intended for this invention.

While numerous inventions are related to protein gelation, they focus primarily on creating protein gel products for food application. The properties of the protein gels are, therefore, limited by the need to preserve qualities such as high nutritional value, edible final product, water-solubility, appearance, mouth-feel and others. For example, Kitabatake, et al. [U.S. Pat. No. 5,416,196] described a method of preparing a heat induced transparent whey protein gel and its product for foodstuffs, but they paid no interest to the opaque aggregate protein gel and harsh chemicals that may strengthen the gel.

For many applications of interest in this invention, i.e. separation and immobilization, it is necessary that the protein gel be porous such that the surface area of the protein gel is increased. Three patents reference to Vieth, et al. (U.S. Pat. No. 3,972,776; U.S. Pat. No. 3,977,941; U.S. Pat. No. 4,601,981) describe the formation of protein membrane that contains cells and enzymes. The mixture of protein and enzymes or cells is cast into membrane of thickness 5-100 microns and dried. The membrane and enzyme/cell are held together by the accumulative effects of multiple hydrogen bonds, salt linkages and van der Waals interactions, although a subsequent cross-linking by dialdehyde is also described to strengthen the membrane. While Vieth, et al. have demonstrated the use of protein gel as an immobilization matrix for cells and enzymes in enzymatic reactions, the membrane is nonporous, and the making and advantage of porous protein gel is not discussed. On top of that, a strong microporous protein gel that functions like a sorbent or separation membrane is completely different from the technology taught by Vieth, et al.

Everhart, et al. [U.S. Pat. No. 5,494,744] teach a method of applying a protein coating to a fibrous fabric. Their final product will be an improved fibrous matrix wherein each fiber is coated with a layer of protein molecules to improve the wettability and hydrophilicity of the fibrous fabric. The morphology of the fibrous fabric is largely preserved. It is different from a protein membrane made completely of protein and is also different from a protein membrane supported on a fibrous fabric. The morphology and characteristic of the protein membrane or supported protein membrane rely completely on the protein gel itself, whereas the fibrous fabric functions mostly as a support for strength.

Damodaran [U.S. Pat. No. 6,310,105] presented a superabsorbent (water) of protein hydrogel made of carboxyl-modified protein (acylated protein), cross-linked by dialdehyde, and washed by ethanol. The superabsorbents adsorb greater than 100 times their dry weight of water and adsorb metal cations. Again, the protein hydrogel made by Damodaran is a homogeneous cross-linked gel that is not microporous.

Protein gel is also made into microspheres for controlled release of active agent. Sutton, et al. (U.S. Pat. No. 5,955,108) described the formation of smooth microspheres for drug delivery by spray-drying a water-soluable protein solution. The microspheres were then linked to drugs or other functional molecules. Sair, et al. (U.S. Pat. No. 4,230,687) described a spherical encapsulation matrix made by emulsifying an active organic agent in a high concentration/viscosity protein solution and then spray-dried. Rosenberg (U.S. Pat. No. 5,601,760) also described a similar spherical encapsulation matrix, but achieved by emulsion method as compared to spray-drying. All of the microspheres described were not designed for separation such as chromatography media.

### BRIEF SUMMARY OF THE INVENTION

It is clear from the discussion above that the prior art does not address or does not adequately address the use of protein gel, particularly microporous protein gel, for separation, immobilization, diagnosis, and artificial organ applications. This invention therefore focuses on the development of said unfulfilled applications.

The objective of this invention is to develop a method for the formation of protein gel matrices that can be used for separation, immobilization, diagnosis, and artificial organ applications. Specifically hollow fiber membranes, supported and non-supported tubular membranes, supported and non-supported flat sheet membranes, continuous bed chromatography media, and protein sorbent of any morphology, size, and shape are disclosed in this invention. This invention further discloses the post-formation modification of the protein gels to enhance their performances by chemical modification, enzymatic modification, physical modification.

### DETAILED DESCRIPTION OF THE INVENTION

The general method of making protein gel matrices for separation, immobilization, diagnosis, and artificial organ applications consists of the following steps:

1. Preparation of a protein solution according to a predetermined formula known to yield desired characteristics.

2. Formation of the protein solution into matrices of desired conformations for separation, immobilization, diagnosis, and artificial organ applications.

3. Post-formation modification of protein matrices to enhance the properties of the matrices.

1) Preparation of Protein Solution

Formulating an appropriate protein solution that could produce a protein gel with desired morphology and characteristics is a very important step. Aggregated protein solution made of 30 wt % whey protein isolate and 0.1M calcium chloride, for example, will produce a micro-porous aggregate protein gel upon heat-induced gelation. The formation of a heat induced protein gel is a two-step process involving the association (aggregation) of protein molecules and the subsequent cross-linking (gelation) of the protein molecules to form a gel. Protein molecules aggregate depending on the electrostatic forces and ionic strength of the protein solution, alterable by the salt content and solution pH. Changing the electrostatic forces and ionic strength will change the size of the protein aggregates therefore changing the morphology of the final aggregate gel. In general, controlling the pH of the protein solution near the isoelectric point of the protein or adding salt into the protein solution will favor protein aggregation and the formation of micro-porous aggregate gel. On the other hand, a dense protein gel can be made with a protein solution that is low on salt content and has a pH away from the isoelectric point of the protein. The strength of the porous and nonporous gel can also be enhanced by increasing the concentration of protein. This gelling behavior of protein is common in many globular proteins such as whey protein, egg white protein, casein, β-lactoglobulin, α-lactalbumin, ovalbumin, blood/bovine serum albumin, immunoglobulin, and others.

Before the gelation process that permanently set the protein gel, the protein molecules exhibiting amphiphilic characteristics will naturally self-assemble at the air-water or solid-water interface to reduce the surface tension. After the heat induced gelation process, this self-assembled protein layer will eventually turn into a dense but thin (about 1 micron) skin layer over the porous aggregate protein gel phase. As a protein membrane (thinly cast protein gel), this dense skin layer will function like an ultra fine sieve (ultrafiltration) that is rendered strength by the layer of microporous gel phase beneath it. This combination of thin dense skin over a porous gel phase also enables the membrane to work like an ultra-fine sieve without requiring an extremely high pressure to drive the fluid through the skin of the membrane since it is thin. Present investigation has indicated that the membrane has a molecular weight cut-off of 1,000 (can retain at least 90 percent of molecules with 1,000 molecular weight or above). While the formation of the dense skin is, in essence, non-avoidable due to the nature of most protein molecules, the porosity of the skin layer can be modified by adding surfactant into the casting solution before the gelation process. Doing so, the self-assembling of protein molecules at the interface is disrupted since the surfactants will be absorbed at the interface preferentially.

The protein molecules used in the preparation of protein solution can be pre-modified chemically or enzymatically to expand the usefulness and flexibility of the protein gel. For example, chemically or enzymatically replacing the amine groups on the protein molecules by carboxyl groups will increase the anionic characteristic of the protein molecules and ultimately the protein gel. Chemically bonding affinity ligands or enzymes onto the protein molecules can produce modified complex protein molecules that, in turn, can be made into specialty membrane. Plasticizers, heat stable enzymes, heat stable cells, polymers, catalysts, activated carbon particles, and others additives can also be blended physically into the protein solution before the gelation to improve the performance of the protein gel. For example, incorporating enzyme (one type of protein) such as α-amylase into the protein gel can be used to form a protein gel with enzymatic activity suitable for bioreaction. Using different proteins or different combinations of proteins in the preparation of protein solution will also produce protein gel of different characteristics.

In summary, the morphology and the characteristics of the protein membrane depend greatly on the protein solution and can be varied by the makeup of the protein solution, including: a) protein type (include modified protein), b) protein concentration, b) salt concentration, c) pH, d) surfactant concentration, e) additive type, and i) additive concentration.

2) Forming Protein Gel Matrix

The fabrication of protein gel matrices, with conformations suited for separation, immobilization, diagnosis, and artificial organ applications, converts the protein solution into usable final forms. These conformations of protein gel include: a) flat sheet protein membrane [Teo & Beitle, 2001], b) tubular protein membrane, c) supported flat sheet protein membrane, d) supported tubular membrane, and e) protein gel sorbent of any size and shape.

The flat sheet protein membrane (a), tubular protein membrane (b), and protein gel sorbent (e), can be made by casting, molding or extruding a protein solution followed by heating under predetermined operating conditions. Supported membrane in tubular (b) and flat sheet (c) forms can be made by casting, coating, or impregnating a porous sheet or porous tube with the protein solution, followed by heating under predetermined operation conditions. The porous sheet and tube to be coated or cast further include the pre-made tubular and flat sheet protein membrane. Spherical form of protein gel sorbent (e) can be made by heat induced emulsion polymerization and spraying drying. Irregular size and shape of protein gel sorbent (e) can be made by breaking a molded or extruded piece of protein gel into smaller pieces.

3) Post Formation Modification

The protein matrices formed can be further strengthened by cross-linking with bifunctional molecules such as dialdehyde. Annealing the protein matrices near the glass transition temperature of the protein matrices will also strengthen the gel. Enzyme, catalyst, affinity ligands and other molecules can also be covalently linked to the protein matrices to impart desired characteristics to the protein matrices.

In conclusion, the technology discussed above has enabled the formation and modification of a protein matrix that is suitable for separation, immobilization, diagnosis, and artificial organ applications. The focus on developing a protein matrix with different morphologies, separation characteristics, sizes, and shapes for said applications distinguishes this invention from other inventions where such concerns are not important. This invention further introduces a novel separation matrix made of protein that is completely different from the traditional polymeric or inorganic based separation matrix. The characteristics of this protein matrix include excellent organic solvent stability, naturally hydrophilic, non-toxic, edible, green fabrication process, easily modifiable (contains plenty of amine, hydroxyl, carboxyl, and other active group), controllable morphology, and others. This protein matrix is suitable for applications including membrane separation, affinity separation, chromatographic separation, enzyme-linked immuno sorbent assay, catalysts immobilization, enzymes immobilization, cells immobilization, electrophoresis, adsorption, medical device, artificial organ, wound protection, and others.

The examples described hereafter illustrate the use of whey protein isolate (WPI) as a protein source for the fabrication of selected protein based separation matrices disclosed in this invention:

### EXAMPLE 1

### Fabrication of Self Supporting WPI Ultrafiltration Membrane

Preparation:

1) Completely dissolve 3 g of WPI in 7 ml of 0.075M CaCl₂ solution (30% WPI and 70% CaCl₂ solution).

2) Centrifuge the mixture at low speed to remove bubbles.

3) Carefully and evenly cast 5 ml of solution into a 0.5 ft by 0.5 ft square on a nonstick flat baking pan.

4) Heat the baking pan in an autoclave at 121° C. for 60 min.

5) Remove the baking pan from the autoclave and quench the membrane in cold water.

6) Peel the membrane from baking pan can keep it in refrigerator until use.

Description:

This membrane has a thin skin layer of 1,000 molecular weight cut-off and a bulk porous membrane phase made of aggregated protein gel. It is also solvent stable.

### EXAMPLE 2

### Fabrication of Supported Tubular WPI ultrafiltration Membrane

Preparation:

1) Completely dissolve 3 g of WPI in 7 ml of 0.075M CaCl₂ solution.

2) Centrifuge the mixture at low speed to remove bubbles.

3) Carefully coat the lumen of a 2-micron porous polypropylene tube with the casting solution.

4) Heat the coated polypropylene tube in an autoclave at 121° C. for 60 min.

5) Remove the tube from the autoclave and quench it in cold water.

Description:

This membrane is similar to the membrane in Example 1 except that this membrane is supported on a porous tube that provides the membrane with greater mechanical strength. The molecular weight cut-off of the membrane remains at 1,000.

### EXAMPLE 3

### Fabrication of Supported Flat Sheet WPI Ultrafiltration Membrane

Similar to Example 1 except that the membrane is cast on a nonwoven fabric support permanently. The fabric provides the membrane with greater mechanical strength, and the molecular weight cut-off of the membrane remains at 1,000.

### EXAMPLE 4

### Fabrication of Microfiltration Membrane

Self supporting, supported tubular, and supported flat sheet ultrafiltration membranes in Examples 1-3, can also be made for microfiltration membrane in a similar way. The only changes is to add 0.5% of Tween 20 (surfactant) into the protein casting solution. The microfiltration membrane formed will not have the thin skin layer, and the pore size of the membrane is about 1 micron.

### EXAMPLE 5

### Fabrication of Cross-Linked Ultrafiltration and Microfiltration Membranes

Cross-linked ultrafiltration and microfiltration membranes can be made by cross-linking the membrane made in Examples 1-4 with formaldehyde. The membranes are cross-linked by submerging the pre-made membranes in Examples 1-4 in 10% formaldehyde solution for 20 hours, followed by rinsing and stabilizing the cross-linked membranes with sodium borohydride. Cross-linked ultrafiltration and microfiltration membranes have greater mechanical strength and are more stable against degradation.

### EXAMPLE 6

### Fabrication of WPI Sorbent

Fill a column with the protein solution made in steps 1 and 2 in Example 1 and heat the whole content of the column in an autoclave at 121° C. for 60 min. A column of porous protein gel with aggregate size of about 1 micron will be formed. This column can be used as continuous bed chromatography sorbent. Protein gel can also be removed from the column and broken into smaller pieces for any adsorption application.

We claim:

1. A method of making protein matrix for separation, immobilization, diagnosis, and artificial organ, comprising the steps of:
a) Preparing a homogeneous protein solution by dissolving the following components in water:
i. Proteins or mixtures of different proteins selected from whey protein isolate, whey protein concentrate, ovalbumin, casine, beta-lactoglobulin, alpha-lactalbumin, or mixture thereof, at a total protein concentration of 5 to 80 weight percents in the final protein solution;
ii. Metal salts or mixture of different metal salts at a total salt concentration of 0.001M to 0.5M in the final protein solution;
iii. Acid, base, or mixtures thereof to change the pH value of the protein solution to between 2 and 11.
b) Degasing the protein mixtures in Step 1a by centrifugation, vacuuming, sonication, or combinations thereof, to remove the trapped air in the protein solution.
c) Extruding, spinning or casting the protein mixtures in Step 1b into a protein matrix of any size and shape at predetermined conditions;
d) Strengthening the protein matrix through a heat treatment or a series of heat treatments, including steam heating and baking, at predetermined temperature or temperatures, pressure or pressures, and humidity or humidities for predetermined length of time to effectively strengthen the protein matrix.

2. The method of making protein matrix in claim 1, wherein said protein in Step 1ai include plant proteins, animal proteins, and plant and animals proteins that have been subjected to chemical modification, enzymatic modification, physical modification, or combined modifications thereof.

3. The method of making protein matrix in claim 1, wherein said metal salt component in Step 1aii is eliminated.

4. The method of making protein matrix in claim 1, wherein said acid, base, or mixture of acid and base component in Step 1aiii is eliminated.

5. The method of making protein matrix in claim 1, wherein said Step 1c is replaced by casting or coating the protein mixtures in Step 1b permanently onto a porous support of any size, shape, and material, including the pre-made protein matrix, at predetermined conditions.

6. The method of making protein matrix in claim 1, wherein said Step 1c is replaced by molding the protein mixtures in Step 1b into any size and shape at predetermined conditions.

7. The method of making protein matrix in claim 1, wherein said Step 1c is replaced by dispersing the protein mixtures into a continuous phase of dry heat, hot steam, solvent, solution, or any combination thereof.

8. The method of making protein matrix in claim 7, wherein said continuous phase of solvent or solution is controlled at predetermined conditions and mixed with additional chemicals including surfactants, cross-linking agents, denaturants, or mixtures thereof.

9. The method of making protein matrix in claim 1, wherein said components of protein solution in Step 1a further includes cross-linking agents, sulfhydryl reagents, chaotropic reagents, ethanol, or mixtures thereof, at a total concentration of 0.5 to 5 weight percents in the final protein solution.

10. The method of making protein matrix in claim 1, wherein said protein solution in Step 1a further includes cells, cell extract, enzymes, antibodies, antigens, polymers, inorganic particles, affinity ligands, catalysts, or mixtures thereof at a total concentration of 0.5 to 50 weight percents.

11. The method of making protein matrix in claim 1, wherein said protein solution in Step 1a further include plasticizers or mixtures of different plasticizers, including glycerol, sorbitol, and polyethylene glycol, at a total plasticizer concentration of 1 to 70 percents in the final protein solution.

12. The method of making protein matrix in claim 1, wherein said protein solution in Step 1a further includes surfactants or mixtures of different surfactants, including phospholipids, polyoxyethylene sorbitan esters, polyoxyethylene alcohols, sodium dodecyl surface, and cetyl-trimethylammonium bromide, at a total surfactant concentration of 0.0001 to 1 percent in the final protein solution;

13. The method of making protein matrix in claim 1 further comprising a chemical treatment step, a series of chemical treatment steps, or a series of chemical treatment and washing steps before, after, or before and after Step 1d.

14. The method of making protein matrix in claim 13, wherein said chemical treatment include covalent and non-covalent modification of protein matrix such as affinity ligands attachment, enzyme immobilization, cross-linking, and combinations thereof.

15. The method of making protein matrix in claims 1 further comprising breaking the final protein matrix into smaller particles.

16. Protein matrix for separation, immobilization, diagnosis, and artificial organ made according to method in claim 1, including supported or non-supported flat sheet membranes, supported or non-supported tubular membranes, hollow fiber membranes, monoliths for continuous bed chromatography, enzyme-linked immuno sorbents, gas sorbents, liquid sorbents, edible sorbents, hemoperfusion sorbents, and surgical sponges.
